(19) United States
(12) Patent Application Publication
(10) Pub. No.: US 2026/0317942 A1
(43) Pub. Date: Oct. 15, 2026

(54) PLATING APPARATUS, AND METHOD OF ESTIMATING STATE IN PLATING APPARATUS

(71) Applicant: EBARA CORPORATION, Tokyo (JP)

(72) Inventors: Ryosuke HIWATASHI, Tokyo (JP); Tsubasa ISHII, Tokyo (JP); Aryan PRIYANSHU, Tokyo (JP); Koichi MASUYA, Tokyo (JP); Masashi SHIMOYAMA, Tokyo (JP)

(21) Appl. No.: 19/565,181

(22) Filed: Mar. 12, 2026

(30) Foreign Application Priority Data
Mar. 14, 2025 (JP) 2025-040915

(51) Int. Cl. C25D 21/12; C25D 17/02; C25D 17/06; G16C 20/30; G16C 20/90

(52) U.S. Cl. CPC C25D 21/12; C25D 17/02; C25D 17/06; G16C 20/30; G16C 20/90

(57) ABSTRACT

To provide a plating apparatus that makes it possible to estimate a current density at a plating-target surface with high precision even if a change in plating conditions occurs. The plating apparatus includes: a sensor configured to measure a potential of a plating solution in a vicinity of a peripheral portion of a substrate held by a substrate holder; and a state estimator configured to calculate, using the measured potential as input, a state estimate representing a current density at the peripheral portion of the substrate, based on an observation model and a state transition model. The state estimator is configured to execute computation based on the observation model by using a first trained neural network model. When a state variable representing the current density at the peripheral portion of the substrate is input, the first trained neural network model is configured to output an estimate representing the potential of the plating solution in the vicinity of the peripheral portion.

### CROSS REFERENCE TO RELATED APPLICATION

This document claims priority to Japanese Patent Application No. 2025-40915, filed Mar. 14, 2025, the entire contents of which are hereby incorporated by reference.

### BACKGROUND OF THE INVENTION

### Field of the Invention

The present invention relates to a plating technique, and in particular to a technique for measuring a film thickness distribution of a plating film.

### Description of the Related Art

In recent years, plating has become one of the important steps in the device manufacturing process. For example, plating is widely adopted for forming fine structures such as wiring of semiconductor integrated circuits and bumps (protruding metal terminals). Control of a film thickness distribution of a plating film (for example, uniformization of the film thickness distribution) is an important factor from the viewpoint of ensuring product performance, reliability, and the like. Techniques for measuring the film thickness distribution fulfill an important role in quality control of the plating film, optimization of the manufacturing process, and the like.

A method of measuring a film thickness distribution of a plating film is disclosed, for example, in Japanese Patent Application Publication No. 2023-160356). This method uses a potential sensor disposed in a vicinity of a peripheral portion of a substrate with a plating-target surface, and a state space model. According to this method, upon measuring a potential with the potential sensor during a formation process of the plating film, the current density of a plating current at the peripheral portion (hereinafter referred to as “peripheral portion current density”) is estimated by executing state estimation processing based on the state space model using the measured potential. Furthermore, a current density at a region inward of the peripheral portion of the substrate is estimated based on the peripheral portion current density. Subsequently, the film thickness distribution of the plating film on the substrate is calculated by using a distribution of the estimated current density (see Japanese Patent Application Publication No. 2023-160356, paragraphs [0043] to [0069]).

During plating processing of forming a plating film on a substrate, various parameters (for example, a position and an orientation of a substrate holder) that define conditions of the forming process of the plating film (that is, plating conditions) may change over time. The plating conditions may change from one plating processing to another (for example, the plating conditions may differ between the plating processing of forming a plating film on a first substrate and the plating processing of forming the plating film on a second substrate). Even if such a change in the plating conditions occurs, it is desirable to perform measurement of a film thickness distribution of the plating film with high precision. When the film thickness distribution of the plating film is estimated from a current density distribution, achieving high-precision measurement of this film thickness distribution requires estimating the current density on the substrate with high precision in response to the change in the plating conditions. This is particularly important when monitoring the film thickness distribution of the plating film in real-time.

### SUMMARY OF INVENTION

In view of the above, an object of the present invention is to provide a plating apparatus, a method of estimating a state in the plating apparatus, and a computer program that make it possible to estimate a current density on a substrate with high precision even if a change in plating conditions occurs.

A plating apparatus according to a first aspect of the present invention includes: a plating bath for accommodating a plating solution; a substrate holder for holding a substrate; an anode disposed in the plating bath so as to be opposite the substrate held by the substrate holder; a sensor configured to measure a potential of the plating solution in a vicinity of a peripheral portion of the substrate held by the substrate holder; and a state estimator configured to calculate, using the measured potential as input, a state estimate representing a current density at the peripheral portion of the substrate, based on an observation model and a state transition model. The state estimator is configured to execute computation based on the observation model by using a first trained neural network model or a first lookup table. When a state variable representing the current density at the peripheral portion is input, the first trained neural network model or the first lookup table is configured to output an estimate representing the potential of the plating solution in the vicinity of the peripheral portion.

A plating apparatus according to a second aspect of the present invention includes: a plating bath for accommodating a plating solution; a substrate holder for holding a substrate; an anode disposed in the plating bath so as to be opposite the substrate held by the substrate holder; a sensor configured to measure a potential of the plating solution in a vicinity of a peripheral portion of the substrate held by the substrate holder; and a state estimator configured to calculate, from the measured potential, a state estimate representing a current density at the peripheral portion of the substrate; and a current density calculator configured to calculate, from the state estimate calculated by the state estimator, a distribution of a current density in an inner region of the substrate located inward of the peripheral portion of the substrate, by using a trained neural network model or a lookup table.

A method according to a third aspect of the present invention is a method of estimating a state in a plating apparatus including a plating bath for accommodating a plating solution, a substrate holder for holding a substrate, an anode disposed in the plating bath so as to be opposite the substrate held by the substrate holder, and a sensor configured to measure a potential of the plating solution in a vicinity of a peripheral portion of the substrate held by the substrate holder, the method including: receiving data of the measured potential from the potential sensor; and calculating, from the measured potential, a state estimate representing a current density at the peripheral portion of the substrate, based on an observation model and a state transition model, wherein the calculating of the state estimate includes executing computation based on the observation model by using a first trained neural network model or a first lookup table, and when a state variable representing the current density at the peripheral portion is input, the first trained neural network model or the first lookup table is configured to output an estimate representing the potential of the plating solution in the vicinity of the peripheral portion.

A method according to a fourth aspect of the present invention is a method of estimating a state in a plating apparatus including a plating bath for accommodating a plating solution, a substrate holder for holding a substrate, an anode disposed in the plating bath so as to be opposite the substrate held by the substrate holder, and a sensor configured to measure a potential of the plating solution in a vicinity of a peripheral portion of the substrate held by the substrate holder, the method including: receiving data of the measured potential from the potential sensor; and calculating, from the measured potential, a state estimate representing a current density at the peripheral portion of the substrate; and calculating, from the calculated state estimate, a distribution of a current density in an inner region of the substrate located inward of the peripheral portion of the substrate, by using a trained neural network model or a lookup table.

A computer program according to a fifth aspect of the present invention includes a plurality of instructions and causes a processor to implement the method according to the fourth aspect or the fifth aspect when the plurality of instructions are executed by the processor.

### BRIEF DESCRIPTION OF DRAWINGS

FIG. 1 is a perspective view showing an overall configuration of a plating apparatus according to a first embodiment of the present invention.

FIG. 2 is a plan view of the plating apparatus of the first embodiment as viewed from above.

FIG. 3 is a schematic configuration diagram of a hardware configuration example that realizes a control module in the first embodiment.

FIG. 4 is a cross-sectional view schematically showing a configuration of a plating module in the first embodiment.

FIG. 5 is a schematic plan view of the substrate.

FIG. 6 is an enlarged view of a surrounding region of a conduit in the plating module of the first embodiment.

FIG. 7 is a schematic diagram of a shield and the substrate of the first embodiment as viewed from below.

FIG. 8 is a functional block diagram showing a schematic configuration of the control module in the first embodiment.

FIG. 9 is a diagram for describing a current density at a position in a peripheral portion of the substrate.

FIG. 10 is a diagram showing a schematic configuration of a first neural network model of the first embodiment.

FIG. 11 is a diagram showing a schematic configuration of a second neural network model of the first embodiment.

FIG. 12 is a flowchart showing an example of a processing procedure for calculating a film thickness distribution of a plating film according to the first embodiment.

FIG. 13 is a functional block diagram showing a schematic configuration of a control module in a second embodiment of the present invention.

FIG. 14 is a diagram showing a schematic configuration of a first lookup table of the second embodiment.

FIG. 15 is a diagram showing a schematic configuration of a second lookup table of the second embodiment.

FIG. 16 is a flowchart showing an example of a processing procedure for calculating the film thickness distribution of the plating film according to the second embodiment.

FIG. 17 is a cross-sectional view schematically showing a configuration of a plating module of a variation of the first embodiment or the second embodiment.

FIG. 18 is a cross-sectional view schematically showing a configuration of a plating module in a third embodiment of the present invention.

### DETAILED DESCRIPTION OF THE PREFERRED EMBODIMENTS

Hereinafter, various embodiments according to the present invention will be described in detail with reference to the drawings. Note that in the drawings, constituent elements denoted by the same reference numerals have the same configurations and functions. Note that an X-axis, a Y-axis, and a Z-axis shown in the drawings are orthogonal to each other.

### First Embodiment

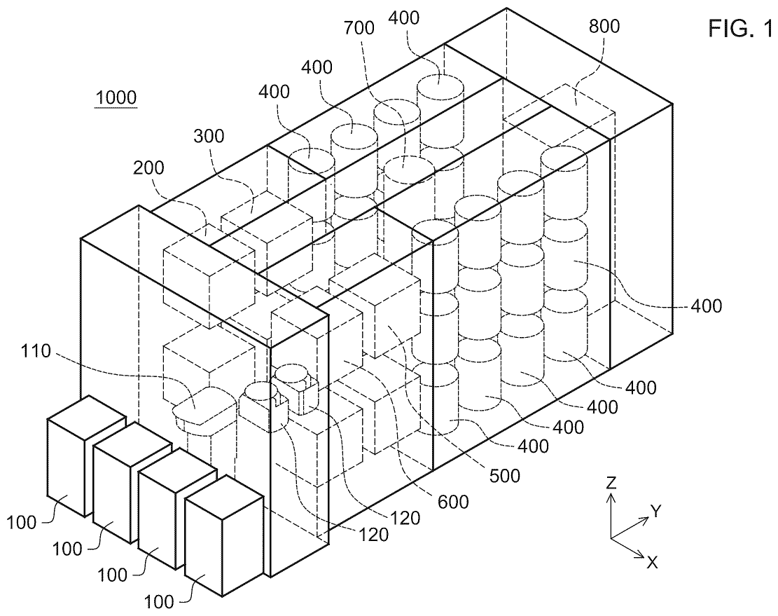

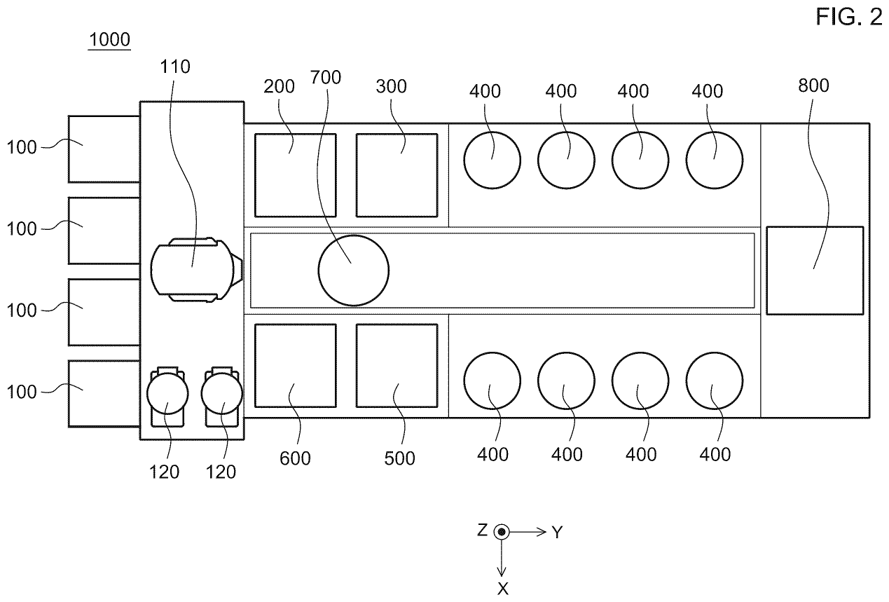

FIG. 1 is a perspective view showing an overall configuration of a plating apparatus 1000 of a first embodiment. FIG. 2 is a plan view of the plating apparatus 1000 of the present embodiment as viewed from above. For convenience of description, in FIG. 2, an upper part of a housing of the plating apparatus 1000 is not shown to allow an internal structure of the plating apparatus 1000 to be visible. As shown in FIGS. 1 and 2, the plating apparatus 1000 includes a load port 100, a transfer robot 110, an aligner 120, a pre-wet module 200, a pre-soak module 300, a plating module 400, a cleaning module 500, a spin rinse dryer 600, a transfer device 700, and the control module 800.

The load port 100 is a module for loading a substrate accommodated in a cassette (not shown) such as a front-opening unified pod (FOUP) into the plating apparatus 1000, unloading the substrate from the plating apparatus 1000 into a cassette, and the like. In the present embodiment, four load ports 100 are disposed side by side along a horizontal direction (X-axis direction), but the number and arrangement of the load ports 100 are not limited thereto and may be freely selected. The transfer robot 110 is a robot for transferring the substrate, and has a function of handing over the substrate between the load port 100, the aligner 120, and the transfer device 700. The transfer robot 110 and the transfer device 700 can hand over the substrate via a temporary stand (not shown) when handing over the substrate between the transfer robot 110 and the transfer device 700.

The aligner 120 is a module for aligning a position of an orientation flat, a notch, and the like of the substrate in a predetermined direction. In the present embodiment, two aligners 120 are disposed side by side along a horizontal direction (Y-axis direction), but the number and arrangement of the aligners 120 are not limited thereto and may be freely selected. The pre-wet module 200 replaces air inside a pattern formed on a surface of the substrate with a processing liquid such as pure water or degassed water, by wetting a plating-target surface of the substrate before plating processing. The pre-wet module 200 can perform pre-wet processing to facilitate supplying a plating solution inside the pattern by replacing the processing liquid inside the pattern with the plating solution (electrolyte solution) during plating. In the present embodiment, two pre-wet modules 200 are disposed side by side along a vertical direction (Z-axis direction), but the number and arrangement of the pre-wet modules 200 are not limited thereto and may be freely selected.

For example, the pre-soak module 300 performs pre-soak processing of etching and removing an oxide film with high electrical resistance present on a surface such as a surface of a seed layer formed on the plating-target surface of the substrate before the plating processing by using a processing liquid such as sulfuric acid or hydrochloric acid, and of cleaning or activating an underlying plating surface. In the present embodiment, two pre-soak modules 300 are disposed side by side along the vertical direction (Z-axis direction), but the number and arrangement of the pre-soak modules 300 are not limited thereto and may be freely selected.

The plating module 400 performs plating processing on the substrate. In the present embodiment, a total of 24 plating modules 400 are arranged. Specifically, twelve plating modules 400 are disposed in a matrix of three rows in the vertical direction (Z-axis direction) and four columns in the horizontal direction (Y-axis direction) on one side of the plating apparatus 1000, and twelve plating modules 400 are disposed in a matrix of three rows in the vertical direction (Z-axis direction) and four columns in the horizontal direction (Y-axis direction) on the other side of the plating apparatus 1000. However, the number and arrangement of the plating modules 400 is not limited thereto and may be freely selected. A specific configuration example of the plating module 400 will be described below.

The cleaning module 500 performs cleaning processing on the substrate for removing unnecessary residue such as plating solution remaining on the substrate after the plating processing. In the present embodiment, two cleaning modules 500 are disposed side by side along the vertical direction (Z-axis direction), but the number and arrangement of the cleaning modules 500 are not limited thereto and may be freely selected. The spin rinse dryer 600 is a module for drying the substrate by rotating the substrate at a high speed after the cleaning processing. In the present embodiment, two spin rinse dryers 600 are disposed side by side along the vertical direction (Z-axis direction), but the number and arrangement of the spin rinse dryers 600 are not limited thereto and may be freely selected. The transfer device 700 is a device for transferring the substrate between the plurality of modules within the plating apparatus 1000.

The control module 800 controls operations and states of the plurality of modules in the plating apparatus 1000. For example, the control module 800 may be implemented as a computer operable by an operator, and it is desirable that the control module 800 includes user interface equipment such as a pointing device and a key input device so that the operator can input information.

Such a control module 800 may be achieved by one computer including one or more processors, or may be achieved by a plurality of computers connected to each other via a communication path. All or part of constituent elements of the control module 800 may be achieved by one or more processors including one or more computation apparatuses (processing units) that execute processing through software or firmware code (plurality of instructions) read from a non-volatile memory (computer-readable recording medium). For example, a CPU (Central Processing Unit), a GPU (Graphics Processing Unit), or an NPU (Neural Network Processing Unit) can be used as the computation apparatus. GPUs and NPUs are computation apparatuses designed to have a structure suitable for computation (for example, tensor computation) based on artificial neural network (ANN), that is, a neural network model (NN model). Alternatively, all or part of the constituent elements of a control module 800 can be achieved by one or more processors having a semiconductor integrated circuit such as an FPGA (Field-Programmable Gate Array). Alternatively, all or part of the constituent elements of the control module 800 may be achieved by one or more processors including a combination of a semiconductor integrated circuit such as an FPGA and computation apparatus such as a CPU or a GPU.

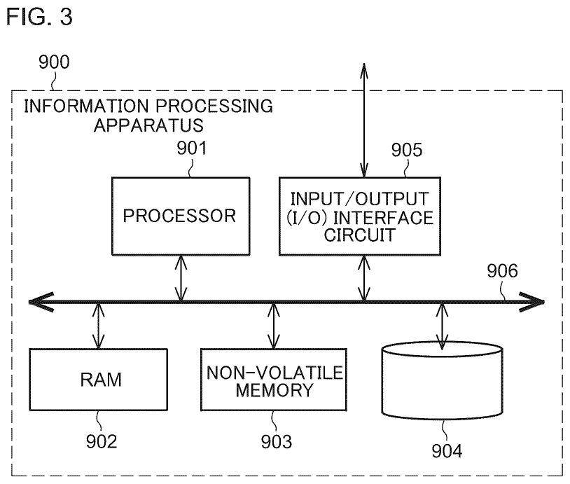

FIG. 3 is a schematic configuration diagram of an information processing apparatus (computer) 900 being a hardware configuration example for realizing the control module 800. The information processing apparatus 900 includes a processor 901, a random access memory (RAM) 902, a non-volatile memory 903, a large-capacity storage 904, an input/output (I/O) interface circuit 905, and a signal path 906. The signal path 906 is a bus for connecting the processor 901, the RAM 902, the non-volatile memory 903, the storage 904, and the input/output (I/O) interface circuit 905 to each other. The RAM 902 is a data storage region used when the processor 901 executes digital signal processing. When a computation apparatus such as a CPU or a GPU is incorporated in the processor 901, the non-volatile memory 903 can have a data storage region in which software code executed by the processor 901 is stored. For example, the input/output (I/O) interface circuit 905 can be connected to user interface equipment (not shown).

Next, an example of a series of plating processing by the plating apparatus 1000 will be described.

First, a substrate accommodated in a cassette is loaded into the load port 100. Subsequently, the transfer robot 110 takes the substrate out of the cassette of the load port 100 and transfers the substrate to the aligner 120. The aligner 120 aligns a position of an orientation flat, a notch, and the like of the substrate in a predetermined direction. The transfer robot 110 hands over the substrate whose direction has been aligned by the aligner 120 to the transfer device 700.

The transfer device 700 transfers the substrate received from the transfer robot 110 to the pre-wet module 200. The pre-wet module 200 performs pre-wet processing on the substrate. The transfer device 700 transports the substrate on which the pre-wet processing has been performed to the pre-soak module 300. The pre-soak module 300 performs pre-soak processing on the substrate. The transfer device 700 transfers the substrate on which the pre-soak processing has been performed to the plating module 400. The plating module 400 performs plating processing on the substrate.

The transfer device 700 transports the substrate on which the plating processing has been performed to the cleaning module 500. The cleaning module 500 performs cleaning processing on the substrate. The transfer device 700 transfers the substrate on which the cleaning processing has been performed to the spin rinse dryer 600. The spin rinse dryer 600 performs drying processing on the substrate. The transfer device 700 hands over the substrate on which the drying processing has been performed to the transfer robot 110. The transfer robot 110 transfers the substrate received from the transfer device 700 to the cassette of the load port 100. Finally, the cassette accommodating the substrate is unloaded from the load port 100.

Note that the configuration of the plating apparatus 1000 described with reference to FIGS. 1 and 2 is merely an example, and the configuration of the plating apparatus 1000 is not limited to the above configuration.

Next, a configuration example of the plating module 400 will be described.

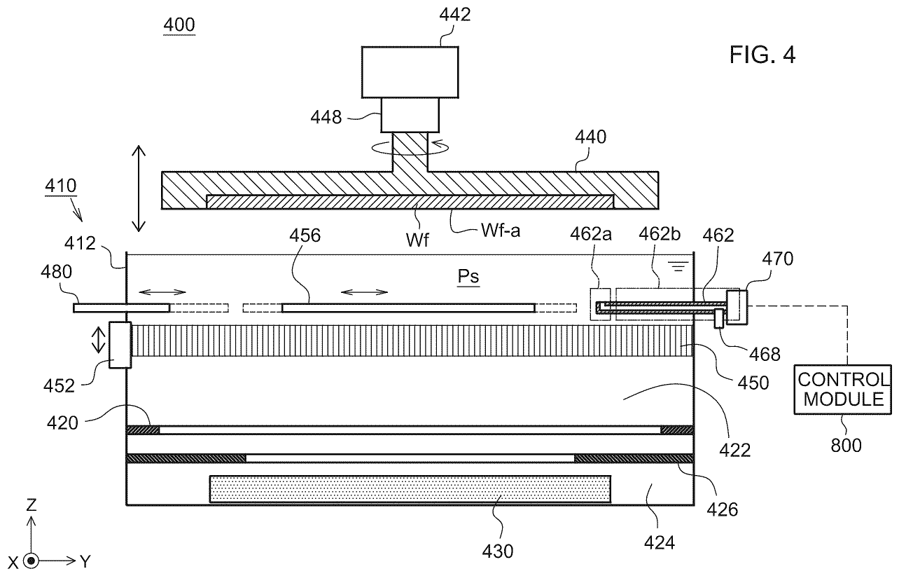

Since the 24 plating modules 400 in the present embodiment have the same configuration, only one plating module 400 will be described. FIG. 4 is a cross-sectional view schematically showing the configuration of the plating module 400 of the first embodiment. As shown in FIG. 4, the plating module 400 includes a plating bath 410 for accommodating the plating solution Ps. The plating bath 410 includes a cylindrical inner tank 412 having an upper surface that is open, and an outer tank (not shown) provided around the inner tank 412 so as to collect plating solution that overflows from an upper edge of the inner tank 412.

The plating module 400 includes a substrate holder 440 for holding the substrate Wf having a plating-target surface Wf−a. As shown in FIG. 4, the substrate holder 440 grips a peripheral portion of the substrate Wf in a state in which the plating-target surface Wf−a is directed toward an opening of the plating bath 410. The plating module 400 includes a lifting mechanism 442 for moving the substrate holder 440 up and down in the Z-axis direction. In one embodiment, the plating module 400 includes a rotation mechanism 448 that causes the substrate holder 440 to rotate about a vertical axis. The lifting mechanism 442 and the rotation mechanism 448 can be achieved by a known mechanism such as a motor. During the plating processing, the lifting mechanism 442 lowers the substrate holder 440 and immerses the plating-target surface Wf−a of the substrate Wf in the plating solution Ps.

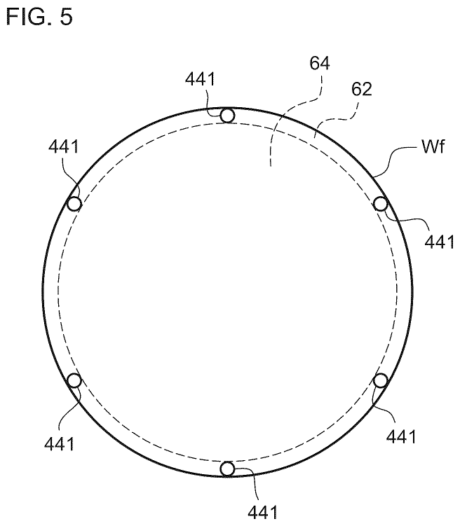

The substrate holder 440 includes one or more power supply electrical contacts for supplying power from a power supply (not shown) to the substrate Wf in a state in which the plating-target surface Wf−a is immersed in the plating solution Ps. FIG. 5 is a schematic plan view of the substrate Wf. The peripheral portion 62 of the substrate Wf is also a portion at which the substrate Wf is gripped by the substrate holder 440. In the example of FIG. 5, the peripheral portion 62 of the substrate Wf has six electrical contacts 441 at equal intervals along a circumferential direction of the substrate Wf, but the number of the electrical contacts 441 is not limited to six. The electrical contacts 441 are connected to a negative terminal of the power supply via electrical wiring (not shown) incorporated embedded inside the substrate holder 440, and a plating current can be supplied to the substrate Wf through the electrical contacts 441. As described below, the control module 800 can calculate the current density distribution of a plating current at the inner region 64 of the substrate Wf located inward of the peripheral portion 62 of the substrate Wf, based on the state estimate representing the current density in the vicinity of the peripheral portion 62 of the substrate Wf.

Referring to FIG. 4, the plating module 400 includes an anode 430 provided on a bottom surface of the inner tank 412. The anode 430 is disposed in the inner tank 412 so as to be opposite the plating-target surface Wf−a of the substrate Wf held by the substrate holder 440. The plating module 400 includes a membrane 420 that separates an internal region of the inner tank 412 into an upper region and a lower region. That is, the inner region of the inner tank 412 is divided by the membrane 420 into a cathode region 422 comparatively close to the plating-target surface Wf−a and an anode region 424 comparatively close to the anode 430. The cathode region 422 and the anode region 424 are each filled with the plating solution Ps. Note that the present embodiment shows an example in which the membrane 420 is provided, but a form in which the membrane 420 is not provided is also possible.

An anode mask 426 for adjusting electrolytic conditions between the anode 430 and the substrate Wf is disposed in the anode region 424. The anode mask 426 is, for example, a substantially plate-like member made of a dielectric material, and is provided facing (or above) the anode 430. The anode mask 426 has an opening through which the current flowing between the anode 430 and the substrate Wf passes. In the present embodiment, the anode mask 426 is configured such that an opening dimension can be changed, and this opening dimension can be adjusted by the control module 800. Here, the opening dimension means a diameter when the opening is circular, and a length of one side or a longest opening width when the opening is polygonal. Note that a known mechanism can be adopted as a means of changing the opening dimension of the anode mask 426. The present embodiment shows an example in which the anode mask 426 is provided, but a form in which the anode mask 426 is not provided is also possible. In the example of FIG. 4, the membrane 420 and the anode mask 426 are provided spatially separated, but the membrane 420 may be provided in the opening of the anode mask 426 instead.

A resistor 450 opposite the membrane 420 is disposed in the cathode region 422. The resistor 450 is a member for achieving uniformity of the plating processing at the plating-target surface Wf−a of the substrate Wf In the present embodiment, the resistor 450 is movable in the vertical direction (Z-axis direction) in the plating bath 410 by a driving mechanism 452, and a position of the resistor 450 can be adjusted by the control module 800. However, a form in which the resistor 450 is not provided in the plating module 400 is also possible. A specific material of the resistor 450 is not particularly limited, but a porous resin such as polyether ether ketone can be used as an example of the material of the resistor 450.

A paddle 456 for stirring the plating solution Ps is provided in a region close to the surface of the substrate Wf in the cathode region 422. The paddle 456 can be made of, for example, titanium (Ti) or resin. The paddle 456 stirs the plating solution so that sufficient metal ions are uniformly supplied to the plating-target surface Wf−a during plating of the substrate Wf, by reciprocating in a direction parallel to the plating-target surface Wf−a of the substrate Wf. The paddle 456 may, for example, move in a direction perpendicular to the plating-target surface Wf−a of the substrate Wf instead. Note that a form in which the paddle 456 is not provided in the plating module 400 is also possible.

A conduit 462 is disposed in the cathode region 422. The conduit 462 is a hollow tube, and can be formed from a resin such as polypropylene (PP) or polyvinyl chloride (PVC). When the resistor 450 is provided in the cathode region 422, the conduit 462 is provided between the substrate Wf and the resistor 450. When the paddle 456 is provided, the conduit 462 is disposed so as not to interfere with the paddle 456. For example, the conduit 462 is preferably disposed at the same height position (the same position in the Z-axis direction) as the paddle 456 and at a position outward of the paddle 456 (in FIG. 4, horizontally outward position).

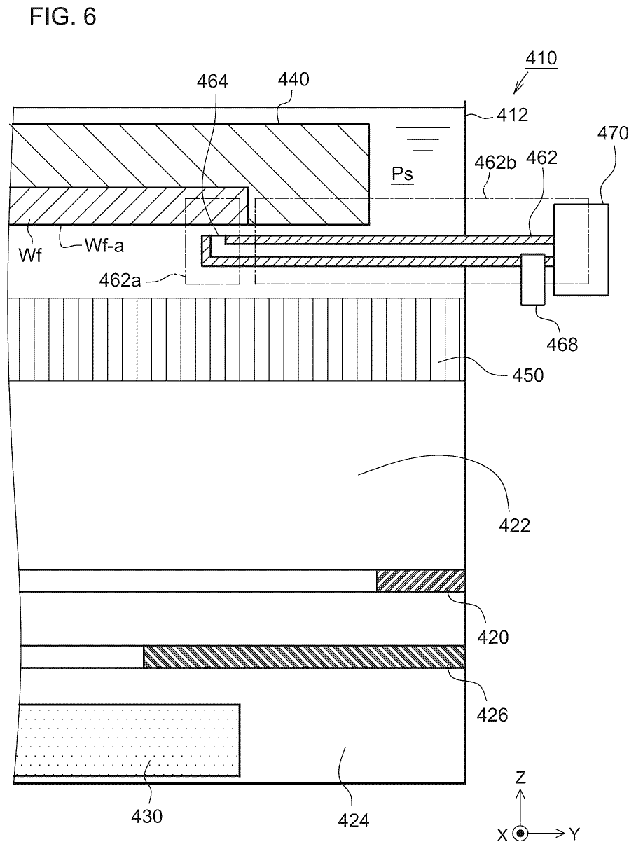

FIG. 6 is an enlarged view of a surrounding region of the conduit 462 in the plating module 400 of the first embodiment. FIG. 6 shows the state in which the substrate holder 440 has descended inside the plating bath 410 and is immersed in the plating solution Ps. As shown in FIGS. 4 and 6, the conduit 462 has an opening end 464 disposed in a region between the substrate Wf and the anode 430. This opening end 464 is positioned between the substrate Wf and the anode 430 in the direction perpendicular to the plating-target surface Wf−a of the substrate Wf (Z-axis direction), and is disposed at a position overlapping with the substrate Wf when viewed from this perpendicular direction. The opening end 464 is preferably disposed in a vicinity of the plating-target surface Wf−a and is opposite the plating-target surface Wf−a. As an example, a distance between the opening end 464 and the plating-target surface Wf−a is several hundred micrometers, several millimeters, or several tens of millimeters. Note that in the examples of FIGS. 4 and 6, the opening end 464 opens in a direction opposite the plating-target surface Wf−a (Z-axis direction), but the present invention is not limited thereto. The opening end 464 may open in a direction perpendicular to a direction in which the substrate Wf and the anode 430 are connected (negative Y-axis direction), or may open in a direction inclined with respect to a normal direction of the plating-target surface Wf−a of the substrate Wf.

In the present embodiment, the conduit 462 extends from the region between the substrate Wf and the anode 430 to a region away from the region between the substrate Wf and the anode 430, and extends outside of the plating bath 410. Hereinafter, as shown in FIGS. 4 and 6, a portion of the conduit 462 disposed in the region between the substrate Wf and the anode 430 is referred to as a “first portion 462a”, and a portion of the conduit 462 disposed in the region away from the region between the substrate Wf and the anode 430 is referred to as a “second portion 462b”. The conduit 462 preferably extends in a direction (Y-axis direction) perpendicular to the direction (Z-axis direction) in which the substrate Wf and the anode 430 are connected. However, the present invention is not limited to such an example, and the conduit 462 may extend in any direction.

An inside of the conduit 462 is filled with the plating solution similar to the cathode region 422. A filling mechanism 468 for filling the conduit 462 with the plating solution may be provided in the conduit 462. Various known mechanisms can be adopted as the filling mechanism 468, and an air vent valve or a mechanism for supplying the plating solution can be adopted as an example. The filling mechanism 468 is provided in the second portion 462b of the conduit 462 as an example.

Note that in FIGS. 4 and 6, one conduit 462 is shown. for clarity, but a plurality of conduits may be provided in the plating bath 410 instead. When the plurality of conduits is provided, an opening end of each conduit may be disposed at different distances from a center of the substrate Wf. When the plurality of conduits are provided, the opening end of each conduit is preferably disposed at a position in which a distance from the plating-target surface Wf−a of the substrate Wf is equal.

A potential sensor 470 is provided at the second portion 462b of the conduit 462. Note that the potential sensor 470 is disposed outside of the plating bath 410 in FIGS. 4 and 6, but may be arranged inside the plating bath 410 instead. The potential sensor 470 has a function of detecting or measuring the potential of the plating solution Ps with which the conduit 462 has been filled. Here, the plating solution Ps in the conduit 462 has substantially the same potential as the plating solution in a vicinity of the opening end 464, and a potential detected by the potential sensor 470 is approximately equal to the potential of the plating solution Ps in the vicinity of the opening end 462a. Thus, the vicinity of the opening end 464 can be set as a pseudo potential detection position for the potential sensor 470, and the potential in the vicinity of the plating-target surface Wf−a can be measured by the potential sensor 470 provided in the second portion 462b of the conduit 462. The potential sensor 470 supplies a measurement signal representing the measured potential to the control module 800.

In one embodiment, a reference potential sensor (not shown) may be provided at a location in the plating bath 410 where a potential change is comparatively small, and a difference between the potential detected by the reference potential sensor and the potential detected by the potential sensor 470 is preferably acquired. The change in the potential measured by the potential sensor 470 is exceedingly small and is therefore easily affected by noise. In order to reduce noise, an independent electrode is preferably installed in the plating solution and directly connected to ground.

The control module 800 has a function of estimating a film thickness distribution of a plating film formed on the substrate Wf, based on the measurement signal representing the potential measured by the potential sensor 470. The control module 800 may detect an endpoint of the plating processing or may predict an amount of time until the endpoint of the plating processing, based on the measurement signal. As an example, the control module 800 may end the plating processing when a film thickness of the plating film reaches a desired thickness, based on the measurement signal. As an example, the control module 800 may calculate a film thickness increase rate of the plating film based on the measurement signal, and predict an amount of time until the plating film reaches the desired thickness, in other words, the amount of time until the endpoint of the plating processing.

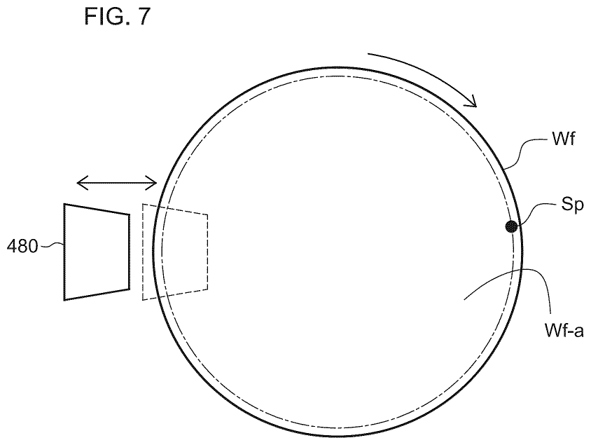

Returning to FIG. 4, in one embodiment, a shield 480 for partially blocking an amount of current flowing from the anode 430 to the substrate Wf is provided in the cathode region 422. The shield 480 is, for example, a substantially plate-like member made of a dielectric material. FIG. 7 is a schematic diagram of the shield 480 and the substrate Wf of the present embodiment as viewed from below (negative Z-axis direction). Note that in FIG. 7, the substrate holder 440 that holds the substrate Wf is omitted. The shield 480 is movable to any position between a shielding position (position indicated by dashed line in FIG. 7) interposed between the plating-target surface Wf−a of the substrate Wf and the anode 430, and a retracted position (position indicated by solid line in FIG. 7) at which the shield 480 is retracted from between the plating-target surface Wf−a and the anode 430. In other words, the shield 480 is movable between the shielding position directly below the plating-target surface Wf−a and the retracted position away from directly below the plating-target surface Wf−a. The position of the shield 480 is controlled by the control module 800 via a driving mechanism (not shown). The movement of the shield 480 can be achieved by a known mechanism such as a motor or a solenoid. In the example of FIG. 7, the shield 480 blocks part of an outer circumferential region of the plating-target surface Wf−a of the substrate Wf in the circumferential direction at the shielding position. In the example of FIG. 7, the shield 480 is formed in a tapered shape that narrows toward the center of the substrate Wf. However, the present invention is not limited to such examples, and a member of any shape determined in advance through experiments or the like can be used as the shield 480.

Next, the plating processing in the plating module 400 of the present embodiment will be described in more detail.

The substrate Wf is exposed to the plating solution by immersing the substrate into the plating solution of the cathode region 422 by using the lifting mechanism 442. The plating module 400 can perform the plating processing on the plating-target surface Wf−a of the substrate Wf by applying a voltage between the anode 430 and the substrate Wf in this state. In one embodiment, the plating processing is performed while rotating the substrate holder 440 by using the rotation mechanism 448. A conductive film (plating film) is deposited on the plating-target surface Wf−a of the substrate Wf through the plating processing. In the present embodiment, during the plating processing, the potential sensor 470 detects or measures in real-time the potential of the plating solution in the vicinity of the peripheral portion of the substrate Wf (for example, at a predetermined detection point Sp shown in FIG. 7). The control module 800 can measure the film thickness of the plating film in real-time, based on the measurement signal representing the potential measured by the potential sensor 470. This allows the film thickness distribution of the plating film formed on the plating-target surface Wf−a of the substrate Wf during the plating processing to be measured and monitored in real-time.

By detecting the potential with the potential sensor 470 accompanying the rotation of the substrate holder 440 (rotation of the substrate Wf), the position detected by the potential sensor 470 can be changed, and the film thickness at a plurality of points in the circumferential direction of the substrate Wf or the entire circumferential direction can be measured.

Note that the plating module 400 may change the rotation speed of the substrate Wf during the plating processing by using the rotation mechanism 448. As an example, the plating module 400 may slowly rotate the substrate Wf for estimating the plating film thickness by using the film thickness estimation function of the control module 800. As an example, the plating module 400 may rotate the substrate Wf at a first rotation speed Rs1 during the plating processing, and the substrate Wf may be rotated at a second rotation speed Rs2 slower than the first rotation speed Rs1 at intervals (for example, every few seconds) while the substrate Wf rotates once or several times. By doing so, even when a sampling period by the potential sensor 470 is short relative to the rotation speed of the substrate Wf, the plating film thickness of the substrate Wf can especially be estimated with precision. Here, the second rotation speed Rs2 may be a tenth of the first rotation speed Rs1.

Data of a change in the film thickness of the plating film measured by using film thickness measurement function of the control module 800 is recorded. In subsequent plating processing, the plating conditions including at least one of the plating current value, the plating time, the opening dimension of the anode mask 426, or the position of the shield 480 can be adjusted with reference to such data. Note that the adjustment of the plating conditions may be performed by a user of the plating apparatus 1000 or through a function of the control module 800. As an example, the adjustment of the plating conditions by the control module 800 may be performed based on a conditional expression determined in advance through experiments, a program, or the like.

The adjustment of the plating conditions may be performed when plating another substrate Wf, or the adjustment of the plating conditions in the current plating processing may be executed in real-time. As an example, the control module 800 may change the plating conditions by adjusting the position of the shield 480.

The control module 800 may adjust the plating conditions in real-time by driving the lifting mechanism 442 or the driving mechanism 452 and adjusting a distance between the substrate Wf and the resistor 450. The distance between the substrate Wf and the resistor 450 comparatively affects the amount of plating formed in a vicinity of an outer circumferential portion of the substrate Wf, while the distance may not comparatively affect the amount of plating formed near the center of the substrate Wf Therefore, as an example, the control module 800 can perform control such that the distance between the substrate Wf and the resistor 450 is reduced when the film thickness of the plating film in the vicinity of the outer circumferential portion of the substrate Wf is greater than a target, and the distance between the substrate Wf and the resistor 450 is increased when the film thickness of the plating film in the vicinity of the outer circumferential portion is smaller than the target. The control module 800 can also perform control such that the distance between the substrate Wf and the resistor 450 increases the longer the shield 480 is in the shielding position, and the distance between the substrate Wf and the resistor 450 decreases the shorter the shield 480 is in the shielding position. By doing so, the amount of plating formed in the vicinity of the outer circumferential portion of the substrate Wf can be adjusted and the uniformity of the plating film formed over the entire substrate Wf can be enhanced.

Furthermore, the control module 800 may adjust the plating conditions in real-time by adjusting the opening dimension of the anode mask 426. As an example, the control module 800 may execute control such that the opening dimension of the anode mask 426 is reduced when the film thickness of the plating film in the vicinity of the outer circumferential portion of the substrate Wf is greater than a target, and the opening dimension of the anode mask 426 is increased when the film thickness of the plating film in the vicinity of the outer circumferential portion is smaller than the target.

Next, a configuration of the control module 800 in the present embodiment will be described in detail below.

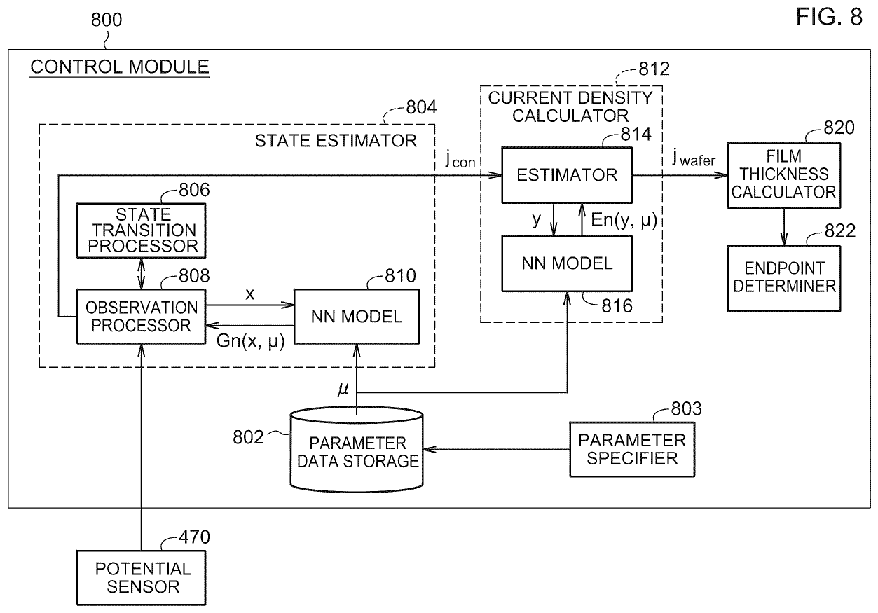

FIG. 8 is a functional block diagram showing a schematic configuration of the control module 800 in the first embodiment. FIG. 8 shows only functional blocks related to the measurement of the film thickness distribution of the plating film among various functions of the control module 800. As shown in FIG. 8, the control module 800 includes a parameter data storage 802, a parameter specifier 803, a state estimator 804, a current density calculator 812, a film thickness calculator 820, and an endpoint determiner 822.

As described above, the potential sensor 470 measures the potential of the plating solution Ps in the vicinity of the peripheral portion of the substrate Wf held by the substrate holder 440 during the plating processing, and outputs the measurement signal representing the measured potential to the state estimator 804. The state estimator 804 can calculate, using a measured potential quantity expressed by the measurement signal as input, a state estimate representing a current density j_{con} at the peripheral portion of the substrate Wf, based on a state space model expressed by the observation model and the state transition model. For convenience of description, the current density at the peripheral portion of the substrate Wf may be hereinafter referred to as “peripheral portion current density”. As shown in FIG. 8, the state estimator 804 includes a state transition processor 806 that performs computation based on the state transition model, a neural network model (NN model) 810, and an observation processor 808 that performs computation based on an observation model by using the NN model 810. The NN model 810 is a trained neural network model (first trained neural network model), and may be achieved by a computer program, or by a hardware configuration such as semiconductor integrated circuit.

The state transition processor 806 and the observation processor 808 can calculate the state estimate representing the peripheral portion current density j_{con} by cooperating with each other and executing state estimation processing using a Kalman filter, based on the input measured potential quantity. The measured potential quantity at each time may be a scalar quantity representing the potential at one point on the peripheral portion of the substrate Wf, or may be a vector quantity representing the potential at a plurality of points on the peripheral portion of the substrate Wf.

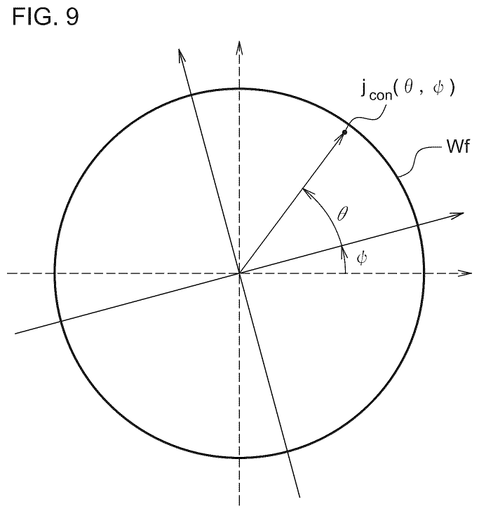

A position on the peripheral portion of the substrate Wf is represented by a pair (θ,ψ) of a rotation angle θ with respect to an electrical contact of the substrate Wf and a rotation angle ψ of the substrate holder 440. FIG. 9 is a diagram for describing the current density j_{con}(θ,ψ) at the position (θ,ψ) in the peripheral portion of the substrate Wf. The peripheral portion current density j_{con}(θ,ψ) is expressed by the following equation (1) using a Fourier series expansion.

$$\begin{matrix}{j}_{con} ( θ , ψ ) = {a}_{0} + {\sum }_{i = 1}^{n} ( {a}_{i} cos i ( θ - ψ ) + {b}_{i} sin i ( θ - ψ ) ) \\ = {a}_{0} + {\sum }_{i = 1}^{n} ( {a}_{i} {b}_{i} ) ( \begin{matrix}cos i ψ & sin i ψ \\ - sin i ψ & cos i ψ\end{matrix} ) ( \begin{matrix}cos i θ \\ sin i θ\end{matrix} ) \\ = {a}_{0} + {\sum }_{i = 1}^{n} ( {a}_{i} {b}_{i} ) {R}_{i} ( ψ ) ( \begin{matrix}cos i θ \\ sin i θ\end{matrix} )\end{matrix} \qquad ( 1 )$$

In this equation, aᵢ and bᵢ (where i is an integer within a range of 0 to n, and n is a positive integer) are Fourier coefficients, and Rᵢ(ψ) is a rotation matrix. The set of Fourier coefficients {aᵢ,bᵢ} can express the peripheral portion current density j_{con}(θ,ψ). The set {aᵢ,bᵢ} is a state variable and can, for example, be expressed in the form of a state vector.

The state transition model is a model that describes a temporal transition of the state variable representing the peripheral portion current density j_{con}. For example, the state transition model that describes a relationship between a state variable at time t and a state variable at time t−1 can be expressed in the form of the following state equation (2) using a state transition function Fᵢ.

$$( \begin{matrix}{a}_{i , t} \\ {b}_{i , t}\end{matrix} ) = {F}_{i} ( \begin{matrix}{a}_{i , t - 1} \\ {b}_{i , t - 1}\end{matrix} ) + {v}_{t - 1} \qquad ( 2 )$$

In this equation, a_{i,t} and b_{i,t} are Fourier coefficients at time t, a_{i,t-1} and b_{i,t-1} are Fourier coefficients at time t−1, and vₜ₋₁ is noise.

The state transition function Fᵢ may be expressed by a linear operator such as a matrix. The state transition function Fᵢ in matrix form is, for example, expressed by the following equations (3) and (4).

$${F}_{i} = \frac{1}{1 + {c}_{i}^{2}} ( \begin{matrix}1 - {c}_{i}^{2} & - 2 {c}_{i} \\ 2 {c}_{i} & 1 - {c}_{i}^{2}\end{matrix} ) \qquad ( 3 )$$

$${c}_{i} = \frac{i ω Δ t}{2} \qquad ( 4 )$$

In these equations, ω is an angular velocity of the rotation of the substrate Wf, and Δt is a time step (that is, a time difference between time t and time t−1). Note that the state equation is not limited to the mathematical expressions expressed by equations (2) to (4). Any state equation can be used provided the state equation can be applied to state estimation processing using a Kalman filter.

In the state estimation processing using the Kalman filter, the state transition processor 806 calculates prior information corresponding to a prior distribution in a Bayesian estimation, based on a predetermined update equation based on the state transition model. For example, the prior information includes a prior state estimate and a prior error covariance matrix.

On the other hand, the observation model is a model that describes a relationship between the measured potential quantity (that is, a sensor observation) and the state variable representing the peripheral portion current density j_{con}. For example, the observation model can be expressed as an observation equation that describes a relationship between a measured potential quantity p at time t and a state variable quantity xₜ representing the peripheral portion current density j_{con} at time t. The observation equation can be expressed in the form of the following equation (5) by using an observation function Gₜ at time t.

$${ϕ}_{t} = {G}_{t} ( \begin{matrix}{a}_{1 , t} \\ {b}_{1 , t} \\ ⋮ \\ {a}_{n , t} \\ {b}_{n , t}\end{matrix} ) + {w}_{t} = {G}_{t} ( {x}_{t} ) + {w}_{t} \qquad ( 5 )$$

In this equation, the observation function Gₜ is a response quantity of the potential sensor 470 with respect to the peripheral portion current density j_{con}, and wₜ is noise.

In the state estimation processing using the Kalman filter, the observation processor 808 calculates posterior information corresponding to a posterior distribution in a Bayesian estimation, based on a predetermined update equation that is based on the observation model, the measured potential quantity, and the prior information each time the measured potential quantity is given. For example, the posterior information includes a posterior state estimate and a posterior error covariance matrix. The posterior state estimate is an estimate of a state at the current time t updated using an actual measured potential quantity and the prior information. The observation processor 808 can output, to the current density calculator 812, the calculated posterior state estimate as a state estimate representing the peripheral portion current density j_{con}.

The NN model 810 can be trained so as to output an estimate (potential estimate) representing the potential of the plating solution Ps in the vicinity of the peripheral portion of the substrate Wf, when the state variable representing the peripheral portion current density j_{con} and the parameter data μ that defines the plating conditions are input. The parameter data μ that defines the plating conditions will be described below.

The observation processor 808 can use the NN model 810 as the observation function G₁, when executing computation based on the observation function Gₜ in the state estimation processing using the Kalman filter. Specifically, when using the NN model 810 as the observation function GE, the observation processor 808 calls the NN model 810 and inputs a variable x to the NN model 810, as shown in FIG. 8. The NN model 810 outputs an inference result Gn(x,μ) in accordance with the input variable x and parameter data p.

Parameters of the plating processing relating to at least one constituent element of the plating module 400 (for example, the plating solution Ps, the plating bath 410, the substrate holder 440, and the anode anode 430) are stored in the parameter data storage 802. The parameters are data that defines conditions of a forming process of the plating film (that is, the plating conditions). Examples of the parameter data include the position and the orientation of the substrate holder 440, the rotation speed of the substrate Wf by the rotation mechanism 448, the type of the plating solution, the electrical conductivity of the plating solution, the polarization gradient of the plating solution, the plating current value, the plating time, the opening dimension of the anode mask 426, the position of the shield 480, and the distance between the substrate Wf and the resistor 450, but the present invention is not limited thereto.

In response to an instruction from the parameter specifier 803, the parameter data storage 802 selects a parameter specified by the instruction from among the parameters stored in the parameter data storage 802, and inputs the parameter data μ indicating a value of the selected parameter to the NN model 810. The parameter specifier 803 has a function of providing, to the parameter data storage 802, an instruction corresponding to information input by the operator through the user interface equipment. As described above, the control module 800 has a control function of adjusting the plating conditions during the plating processing in accordance with the film thickness distribution of the plating film that is measured or estimated. The parameter specifier 803 can provide, to the parameter data storage 802, an instruction specifying the parameters that define the plating conditions adjusted by this control function.

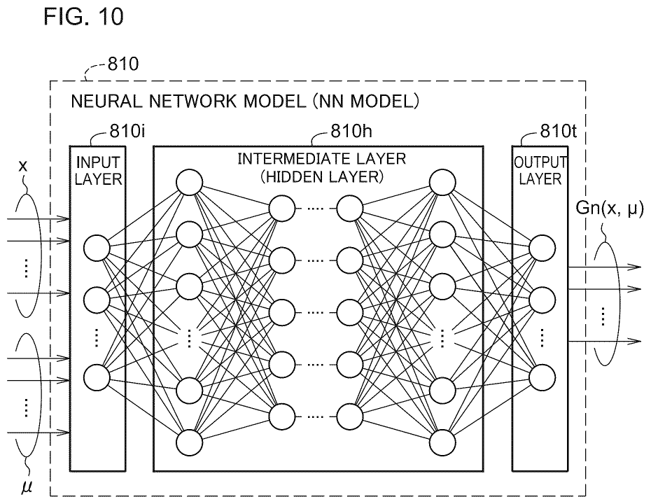

FIG. 10 is a diagram showing a schematic configuration of the NN model 810 of the present embodiment. As shown in FIG. 10, the NN model 810 is a hierarchical artificial neural network including an input layer 810i, an intermediate layer (hidden layer) 810h, and an output layer 810t. The NN model 810 is implemented as a deep learning model. The input layer 810i is configured to accept the parameter data p provided from the parameter data storage 802, and the state variable x. The intermediate layer 810h is configured to couple the input layer 810i and the output layer 810t, and executes inference computation based on the input state variable x and parameter data p. The output layer 810t is configured to output the inference result Gn(x,μ) being a result of this inference computation. The NN model 810 may include an existing feedforward neural network, a convolutional neural network (CNN), a recurrent neural network such as long short-term memory (LSTM), or a transformer with an attention mechanism.

The NN model 810 is trained using supervised learning, unsupervised learning, or semi-supervised learning, in accordance with a known machine learning algorithm. A backpropagation learning algorithm including error backpropagation and gradient descent can be used as the machine learning algorithm. For example, stochastic gradient descent (SGD), momentum SGD, or adaptive moment estimation (Adam) can be used as the gradient descent method, but the present invention is not limited thereto.

Next, referring to FIG. 8, the current density calculator 812 receives supply of the state estimate representing the peripheral portion current density j_{con} calculated by the state estimator 804. The current density calculator 812 calculates, from the state estimate, a current density j_{wafer} in the inner region 64 (FIG. 5) of the substrate Wf located inward of the peripheral portion 62 of the substrate Wf. For convenience of description, the current density in the inner region 64 may be hereinafter referred to as “plating current density”. Specifically, the current density calculator 812 includes an estimator 814 and a neural network model (NN model) 816 as a second trained neural network model (NN model). The estimator 814 calculates, from the state estimate, the plating current density j_{wafer} by using the NN model 816. The NN model 816 may be achieved by a computer program, or by a hardware configuration such as semiconductor integrated circuit.

The NN model 816 outputs an inference result En(y,μ) representing the plating current density j_{wafer}, when a state variable y representing the peripheral portion current density j_{con} and the parameter data μ that defines the plating conditions are input. As shown in FIG. 8, the estimator 814 calls the NN model 816 and inputs the variable y to the NN model 814. The NN model 816 outputs the inference result En(y,μ) in accordance with the input variable x and parameter data p. The estimator 814 can output the inference result En(y,μ) as the estimated plating current density j_{wafer} to the film thickness calculator 820.

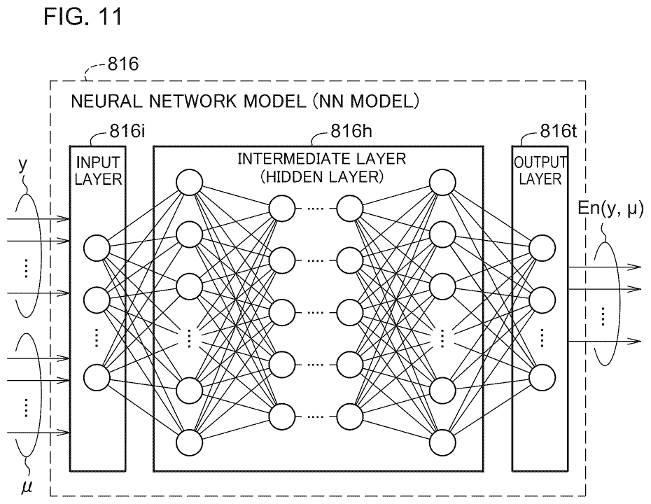

FIG. 11 is a diagram showing a schematic configuration of the NN model 816 of the present embodiment. As shown in FIG. 11, the NN model 816 is a hierarchical artificial neural network including an input layer 816i, an intermediate layer (hidden layer) 816h, and an output layer 816t. The NN model 816 is implemented as a deep learning model. The input layer 816i is configured to accept the parameter data p provided from the parameter data storage 802, and the state variable y. The intermediate layer 816h is configured to couple the input layer 816i and the output layer 816t, and executes inference computation based on the input state variable y and parameter data μ. The output layer 816t outputs the inference result En(y,μ) being a result of this inference computation. The NN model 816 may be configured to include an existing feedforward neural network, a convolutional neural network (CNN), a recurrent neural network such as LSTM, or a transformer with an attention mechanism.

The NN model 816 is trained using supervised learning, unsupervised learning, or semi-supervised learning, in accordance with a known machine learning algorithm. A backpropagation learning algorithm including an error backpropagation method and a gradient descent method can be used as the machine learning algorithm. For example, stochastic gradient descent (SGD), a momentum method (momentum SGD), or an adaptive moment estimation method (Adam) can be used as the gradient descent method, but the present invention is not limited thereto.

Next, referring to FIG. 8, the film thickness calculator 820 calculates the film thickness distribution of the plating film formed on the substrate Wf, based on a plating current density j_{wafer}(k,t) obtained from the current density calculator 812. Here, t is the current time, and k is a number indicating the position of the inner region 64 on the substrate Wf In one embodiment, the film thickness calculator 820 can calculate a deposition rate v(k,t) and a film thickness distribution w(k,t) of the plating film at a position k on the substrate Wf and at the current time t by using the following equations (6) and (7).

$$v ( k , t ) = \frac{M}{zF ρ} {j}_{Wafer} ( k , t ) \qquad ( 6 )$$

$$w ( k , t ) = {\sum }_{q = 0}^{t} v ( k , q ) Δ t \qquad ( 7 )$$

In these equations, M is a molecular weight of the plating deposited on the substrate Wf, ρ is a density of the plating deposited on the substrate Wf, z is a valence of a plating reaction, and F is the Faraday constant. The film thickness calculator 820 may calculate a film thickness w(k,T) at an end time of the plating processing (time q=T) instead of the current film thickness distribution w(k,t), by predicting a future plating current density and deposition rate using the above state equation.

The endpoint determiner 822 determines an endpoint of the plating processing for the substrate Wf, based on the film thickness distribution w(k,t) of the plating film obtained by the film thickness calculator 820. For example, the endpoint determiner 822 may end the plating processing when the estimated current film thickness distribution w(k,t) reaches a desired thickness distribution, or may predict the time to the endpoint of the plating processing based on the estimated current film thickness w(k,t) and the predicted future deposition rate v(k,s) (s=t, . . . , T).

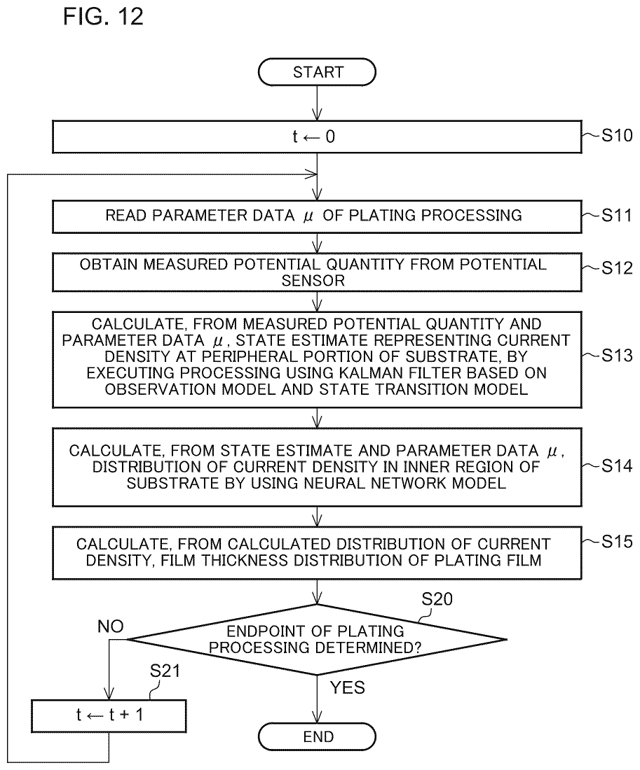

Next, a processing procedure by the above control module 800 will be described hereinafter. FIG. 12 is a flowchart showing an example of a processing procedure for calculating the film thickness distribution of the plating film.

Referring to FIG. 12, the state estimator 804 first initializes time t (step S10). Subsequently, the state estimator 804 reads the parameter data μ supplied from the parameter data storage 802 (step S1), and then obtains the measured potential quantity from the potential sensor 470 (step S12). Here, an order of steps S11 and S12 may be reversed.

Subsequently, the state estimator 804 calculates, from the measured potential quantity and parameter μ, the state estimate representing the current density j_{con} at the peripheral portion of the substrate Wf, by executing the state estimation processing using the Kalman filter based on the observation model and the state transition model (step S13). At this time, the computation based on the observation model is performed using the NN model 810.

Next, the current density calculator 812 calculates, from the state estimate and the parameter data μ, the distribution of the current density (plating current density) j_{wafer} in the inner region of the substrate Wf by using the NN model 816 (step S14). Next, the film thickness calculator 820 calculates the film thickness distribution w(k,t) of the plating film formed on the substrate Wf, based on the plating current density j_{wafer} obtained from the current density calculator 812 (step S15). As described above, the endpoint determiner 822 determines the endpoint of the plating processing for the substrate Wf has been detected, based on the film thickness distribution w(k,t) of the plating film obtained by the film thickness calculator 820 (step S20). Upon determining that the endpoint of the plating processing has been detected (YES in step S20), the control module 800 ends the plating processing. On the other hand, when the endpoint of the plating processing is not detected (NO in step S20), the control module 800 increments time t (step S21) and repeatedly executes the processing from step S11 onward.

As described above, according to the first embodiment, the state estimator 804 of the control module 800 calculates, using the potential measured by the potential sensor 470 as input, the state estimate representing the current density j_{con} at the peripheral portion of the substrate Wf, based on the observation model and the state transition model, wherein the computation based on the observation model is executed using the NN model 810. The NN model 810 is trained so as to output the estimate representing the potential of the plating solution Ps in the vicinity of the peripheral portion, when the state variable representing the current density at the peripheral portion of the substrate Wf is input. The NN model 810 can learn a time-series change in the measured potential quantity in accordance with a change in the plating conditions during the plating processing through training data for machine learning. When a plurality of instances of the plating processing operations are executed consecutively, a change in the plating conditions may occur between one plating processing and another plating processing (for example, the plating conditions may change between the plating processing of forming a plating film on a first substrate and the plating processing of forming the plating film on a second substrate). The NN model 810 can also learn a time-series change in the measured potential quantity in accordance with a change in the plating conditions between such plating processing through training data for machine learning. Therefore, even if a change in the plating conditions occurs, the state estimate representing the current density j_{con}. at the peripheral portion can be calculated with high precision. This enhances estimation precision of the current density j_{wafer} at the plating-target surface Wf−a, and also enhances estimation precision of the film thickness distribution w(k,t) of the plating film. Note that the NN model 810 can be trained so as to learn both a change in the plating conditions based on the parameter data μ and a temporal change in the plating conditions not based on the parameter data μ.

Since the state estimator 804 can sequentially calculate the state estimate representing the current density j_{con} by executing the state estimation processing using the Kalman filter based on the state transition model and the observation model, a highly reliable film thickness distribution w(k,t) can be obtained in real-time. The film thickness distribution of a plating film is conventionally calculated by numerical analysis using computationally intensive numerical simulations, but this method requires many computational resources for performing high-speed calculation. In contrast, the state estimator 804 of the present embodiment can perform high-precision film thickness distribution calculation with high real-time performance using relatively few computational resources.

The current density calculator 812 of the control module 800 can calculate, from the state estimate representing the current density j_{con}, the plating current density j_{wafer} by using the NN model 816. The NN model 816 can learn a time-series change in the peripheral portion current density j_{con} in accordance with a change in the plating conditions during the plating processing through training data for machine learning. When a plurality of instances of the plating processing operations are executed consecutively, a change in the plating conditions may occur between one plating processing and another plating processing (for example, the plating conditions may change between the plating processing of forming a plating film on a first substrate and the plating processing of forming the plating film on a second substrate). The NN model 816 can also learn a time-series change in the peripheral portion current density j_{con} in accordance with a change in the plating conditions between such plating processing through training data for machine learning. Therefore, even if a change in the plating conditions occurs, the current density calculator 812 can estimate the current density j_{wafer} with high precision. This enhances estimation precision of the film thickness distribution w(k,t) of the plating film. Note that the NN model 816 can be trained so as to learn both a change in the plating conditions based on the parameter data μ and a temporal change in the plating conditions not based on the parameter data μ.

### Second Embodiment

Next, a configuration of a control module in a second embodiment will be described in detail below.

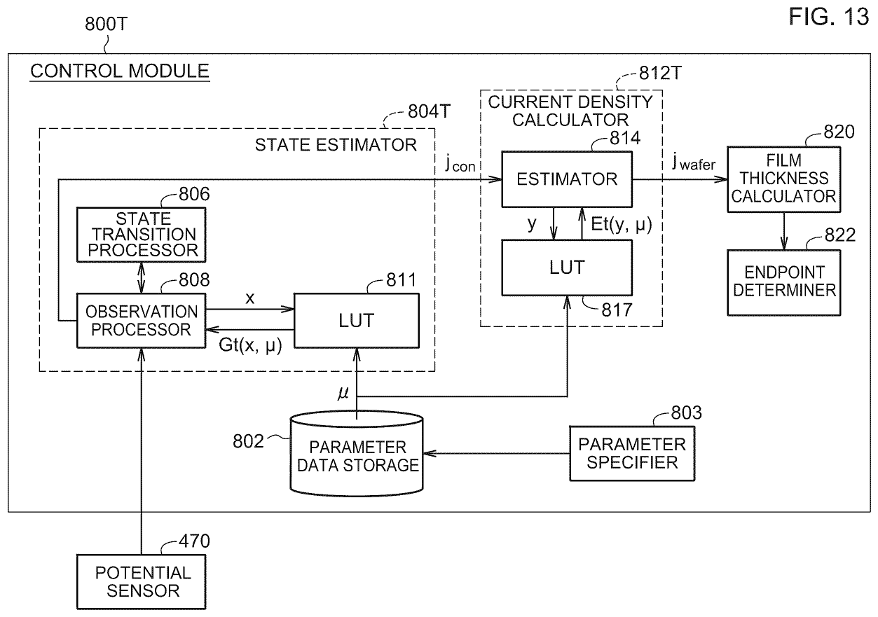

FIG. 13 is a functional block diagram showing a schematic configuration of a control module 800T in the second embodiment. The second embodiment is substantially the same as the first embodiment, except that the control module 800T of the present embodiment is used instead of the control module 800 of the first embodiment.

FIG. 13 shows only functional blocks related to the measurement of the film thickness distribution of the plating film among various functions of the control module 800T. As shown in FIG. 13, the control module 800T includes the parameter data storage 802, the parameter specifier 803, a state estimator 804T, a current density calculator 812T, the film thickness calculator 820, and the endpoint determiner 822. In the control module 800T, the parameter data storage 802, the parameter specifier 803, the film thickness calculator 820, and the endpoint determiner 822 are the same as the functions of the control module 800 of the first embodiment.

The state estimator 804T of the present embodiment has the same function as the state estimator 804 of the first embodiment, except that a lookup table (LUT) 811 is used instead of the above NN model 810. The state estimator 804T can calculate, using the measured potential quantity expressed by the measurement signal obtained from the potential sensor 470 as input, the state estimate representing the current density j_{con} at the peripheral portion of the substrate Wf, based on the state space model expressed by the observation model and the state transition model. As shown in FIG. 13, the state estimator 804T includes the state transition processor 806 that performs computation based on the state transition model, the LUT (first lookup table) 811, and the observation processor 808 that performs computation based on the observation model by using the LUT 811. In the state estimation processing using the Kalman filter, the observation processor 808 of the state estimator 804T can use the LUT 811 as the observation function Gₜ, when executing computation based on the observation function Gₜ of equation (5).

The LUT 811 is a computation device having a data structure in which a correspondence relationship is stored in advance, the correspondence relationship being between the parameter data μ provided from the parameter data storage 802 as first input to the LUT 811, the state variable representing the current density j_{con} at the peripheral portion of the substrate Wf as second input to the LUT 811, and the estimate (potential estimate) representing the potential of the plating solution Ps in the vicinity of the peripheral portion of the substrate Wf as output of the LUT 811.

As described above, in response to an instruction from the parameter specifier 803, the parameter data storage 802 selects a parameter specified by the instruction from among the parameters stored in the parameter data storage 802, and inputs the parameter data μ indicating a value of the selected parameter to the LUT 811. The parameter specifier 803 has a function of providing, to the parameter data storage 802, an instruction corresponding to information input by the operator through the user interface equipment. The control module 800T has a control function of adjusting the plating conditions during the plating processing in accordance with the film thickness distribution of the plating film that is measured or estimated. The parameter specifier 803 can provide, to the parameter data storage 802, an instruction specifying the parameters that define the plating conditions adjusted by this control function.

When using the LUT 811 as the observation function Gₜ of the observation model, the observation processor 808 inputs the state variable x to the LUT 811, as shown in FIG. 13. The LUT 811 can output computation data Gt(x,μ) in accordance with the input state variable x and parameter data μ.

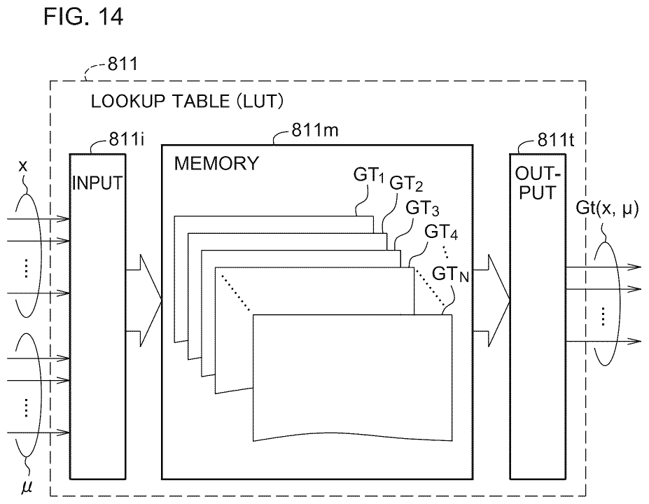

FIG. 14 is a diagram showing a schematic configuration of the LUT 811 of the present embodiment. As shown in FIG. 14, the LUT 811 includes input 811i, a memory 811m, and output 811t. N datasets GT₁, GT₂, GT₃, GT₄, . . . , GT_{N} are stored in the memory 811m in advance. Here, N is a positive integer. For example, N datasets each corresponding to a parameter set that defines plating conditions for several tens of thousands to several millions of cases may be stored as the datasets GT₁ to GT_{N}.

For example, the training data used during the machine learning of the NN model 810 according to the first embodiment can be stored in advance in the memory 811m as the datasets GT₁ to GT_{N}. The training data used for supervised learning includes a pair of input data for the NN model 810 and supervisory data corresponding thereto (output data with correct labels). Such a pair of datasets can be stored in advance in the memory 811m.

When the parameter data μ that defines the plating conditions and the state variable x are input, the input 811i decodes the parameter data μ and the state variable x, and generates a read address that specifies a memory cell in which the computation data Gt(x,μ) corresponding to the state variable x is stored from among a memory cell group in which the dataset GTₙ conforming to the plating conditions is stored. The memory 811m reads the computation data Gt(x,μ) from the memory cell specified by the generated read address. The output 811t can output the read computation data Gt(x,μ).

Next, referring to FIG. 13, the current density calculator 812T receives supply of the state estimate representing the peripheral portion current density j_{con} calculated by the state estimator 804T. The current density calculator 812T is configured to calculate, from the state estimate, the plating current density j_{wafer} in the inner region 64 (FIG. 5) of the substrate Wf, by using a lookup table (LUT) 817. Specifically, the current density calculator 812T includes the estimator 814 and the LUT 817 as a second lookup table. The estimator 814 is configured to calculate, from the state estimate, the plating current density j_{wafer} by using the LUT 817.

The LUT 817 of the current density calculator 812T is configured to output computation data Et(y,μ) representing the plating current density j_{wafer}, when the variable y representing the peripheral portion current density j_{con} and the parameter data μ that defines the plating conditions are input. As shown in FIG. 13, the estimator 814 inputs the variable y to the LUT 817. The LUT 817 can output the computation data Et(y,μ) in accordance with the input state variable y and parameter data μ. The estimator 814 can output the computation data Et(y,μ) as the estimated plating current density j_{wafer} to the film thickness calculator 820.

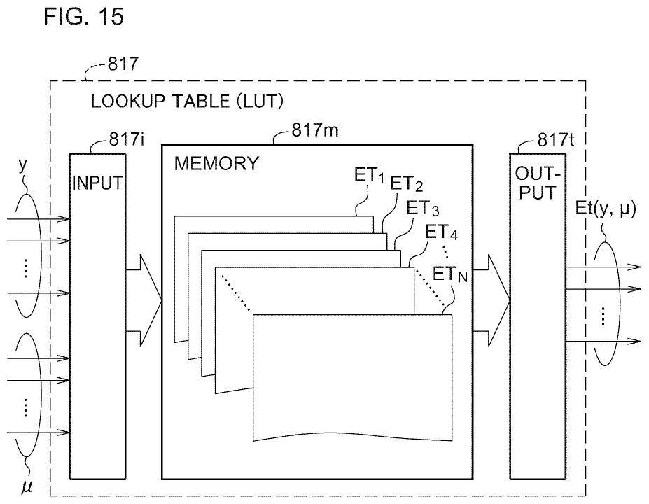

FIG. 15 is a diagram showing a schematic configuration of the LUT 817 of the present embodiment. As shown in FIG. 15, the LUT 817 includes input 817i, a memory 817m, and output 817t. N datasets ET₁, ET₂, ET₃, ET₄, . . . , ET_{N} are stored in the memory 817m in advance. Here, N is a positive integer. For example, N datasets each corresponding to a parameter set that defines plating conditions for several tens of thousands to several millions of cases may be stored as the datasets ET₁ to ET_{N}.

For example, the training data used during the machine learning of the NN model 816 according to the first embodiment can be stored in advance in the memory 817m as the datasets ET₁ to ET_{N}. The training data used for supervised learning includes a pair of input data for the NN model 816 and supervisory data corresponding thereto (output data with correct labels). Such a pair of datasets can be stored in advance in the memory 817m.

When the parameter data μ that defines the plating conditions and the variable y are input, the input 817i decodes the parameter data μ and the state variable y, and generates a read address that specifies a memory cell in which the computation data Et(y,μ) corresponding to the variable y is stored from among a memory cell group in which the dataset ET₁ conforming to the plating conditions is stored. The memory 817m reads the computation data Et(y,μ) from the memory cell specified by the generated read address. The output 817t can output the read computation data Et(y,μ).

Next, referring to FIG. 13, the film thickness calculator 820 calculates the film thickness distribution of the plating film formed on the substrate Wf, based on the plating current density j_{wafer}(k,t) obtained from the current density calculator 812T, similar to the case in the first embodiment. Here, t is the current time, and k is a number indicating the position of the inner region 64 on the substrate Wf In one embodiment, the film thickness calculator 820 can calculate the deposition rate v(k,t) and the film thickness distribution w(k,t) of the plating film at the position k on the substrate Wf and at the current time t by using the above equations (6) and (7). The film thickness calculator 820 may calculate the film thickness w(k,T) at the end time of the plating processing (time q=T) instead of the current film thickness distribution w(k,t), by predicting a future plating current density and deposition rate using the above state equation.

The endpoint determiner 822 determines the endpoint of the plating processing for the substrate Wf, based on the film thickness distribution w(k,t) of the plating film obtained by the film thickness calculator 820. For example, the endpoint determiner 822 may end the plating processing when the estimated current film thickness distribution w(k,t) reaches the desired thickness distribution, or may predict the time to the endpoint of the plating processing based on the estimated current film thickness w(k,t) and the predicted future deposition rate v(k,s) (s=t, . . . , T).

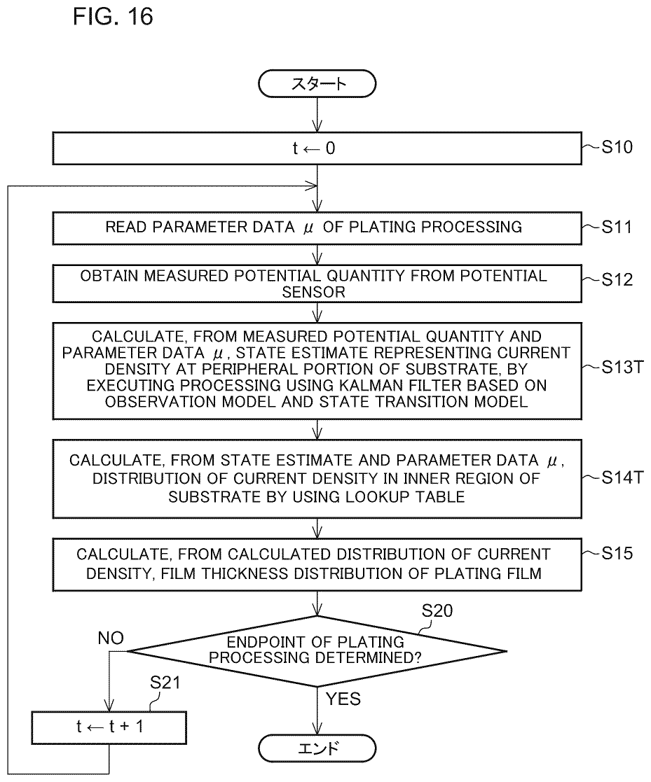

Next, a processing procedure by the above control module 800T will be described hereinafter. FIG. 16 is a flowchart showing an example of a processing procedure for calculating the film thickness distribution of the plating film.

Referring to FIG. 16, the state estimator 804T initializes time t (step S10), reads the parameter data μ supplied from the parameter data storage 802 (step S11), and obtains the measured potential quantity from the potential sensor 470 (step S12). Here, an order of steps S11 and S12 may be reversed.

Subsequently, the state estimator 804T calculates, from the measured potential quantity and parameter μ, the state estimate representing the current density j_{con} at the peripheral portion of the substrate Wf, by executing the state estimation processing using the Kalman filter based on the observation model and the state transition model (step S13T). At this time, the computation based on the observation model is performed using the LUT 811.

Next, the current density calculator 812T calculates, from the state estimate and the parameter data μ, the distribution of the current density (plating current density) j_{wafer} in the inner region of the substrate Wf by using the LUT 817 (step S14T). Next, the film thickness calculator 820 calculates the film thickness distribution w(k,t) of the plating film formed on the substrate Wf, based on the plating current density j_{wafer} obtained from the current density calculator 812T (step S15). The endpoint determiner 822 determines whether the endpoint of the plating processing for the substrate Wf has been detected, based on the film thickness distribution w(k,t) of the plating film obtained by the film thickness calculator 820 (step S20). Upon determining that the endpoint of the plating processing has been detected (YES in step S20), the control module 800T ends the plating processing. On the other hand, when the endpoint of the plating processing is not detected (NO in step S20), the control module 800T increments time t (step S21) and repeatedly executes the processing from step S11 onward.

As described above, according to the second embodiment, the state estimator 804T of the control module 800T is configured to calculate, using the potential measured by the potential sensor 470 as input, the state estimate representing the current density j_{con} at the peripheral portion of the substrate Wf, based on the observation model and the state transition model, wherein the computation based on the observation model is executed using the LUT 811. The LUT 811 is configured to output the estimate representing the potential of the plating solution Ps in the vicinity of the peripheral portion, when the state variable representing the current density at the peripheral portion of the substrate Wf is input. The LUT 811 can store a number of datasets that can flexibly accommodate both a change in the plating conditions during the plating processing and a change in the plating conditions between different instances of the plating processing. Therefore, even if a change in the plating conditions occurs, the state estimate representing the current density j_{con} at the peripheral portion can be calculated with high precision. This enhances estimation precision of the current density j_{wafer} at the plating-target surface Wf−a, and also enhances estimation precision of the film thickness distribution w(k,t) of the plating film.

Since the state estimator 804T can sequentially calculate the state estimate representing the current density j_{con} by executing the state estimation processing using the Kalman filter based on the state transition model and the observation model, a highly reliable film thickness distribution w(k,t) can be obtained in real-time.

The current density calculator 812T of the control module 800T can calculate, from the state estimate representing the current density j_{con}, the plating current density j_{wafer} by using the LUT 817. The LUT 817 can store a number of datasets that can flexibly accommodate both a change in the plating conditions during the plating processing and a change in the plating conditions between different instances of the plating processing. Therefore, even if a change in the plating conditions occurs, the current density calculator 812T can estimate the current density j_{wafer} with high precision. This enhances estimation precision of the film thickness distribution w(k,t) of the plating film.

### Variations

A form in which the current density calculator 812T of the second embodiment is used instead of the current density calculator 812 is also possible as a first variation of the first embodiment. A form in which the state estimator 804T of the second embodiment is used instead of the state estimator 804 is also possible as a second variation of the first embodiment.

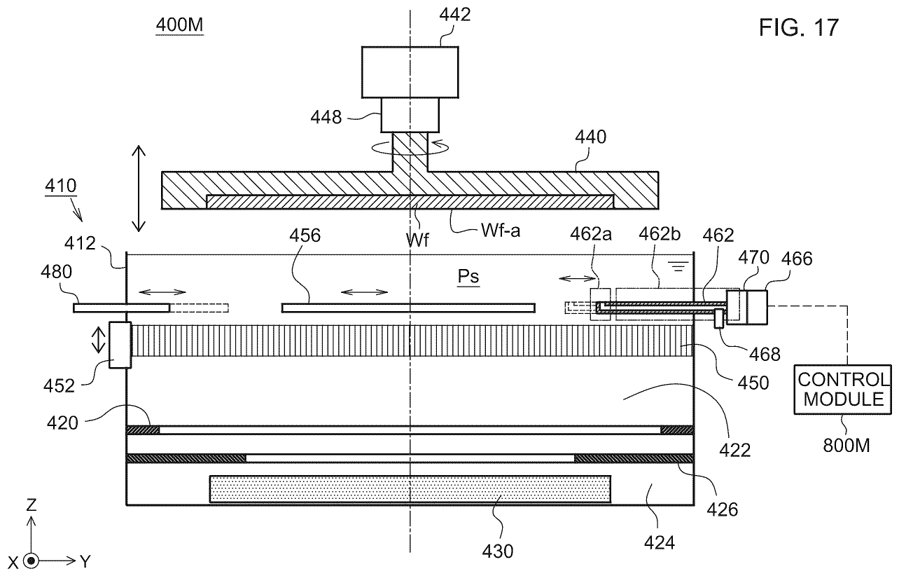

Furthermore, FIG. 17 is a cross-sectional view schematically showing a configuration of a plating module 400M of a variation of the first embodiment or the second embodiment. With respect to the plating module 400M of this variation, portions that overlap with those of the plating module 400 of the first embodiment are denoted by the same reference numerals, and description thereof is omitted. A control module 800M has the same functions as one of the control module 800 of the first embodiment or the control module 800T of the second embodiment. In the plating module 400M of this variation, the conduit 462 is movable by a driving mechanism 466. The driving mechanism 466 is controlled by the control module 800M. The control module 800M can adjust a position of the opening end 464 of the conduit 462 (see FIG. 6), by controlling an operation of the driving mechanism 466. The driving mechanism 466 can be achieved by a known mechanism such as a motor or a solenoid. As described above, since the potential in the conduit 462 detected by potential sensor 470 is roughly equal to the potential in the vicinity of the opening end 464, the pseudo detection position for the potential sensor 470 can be changed by adjusting the position of the opening end 464 of the conduit 462 with the driving mechanism 466. Noted that while not limited, the driving mechanism 466 may have a function of moving the potential sensor 470 along a radial direction of the substrate Wf.

### Third Embodiment

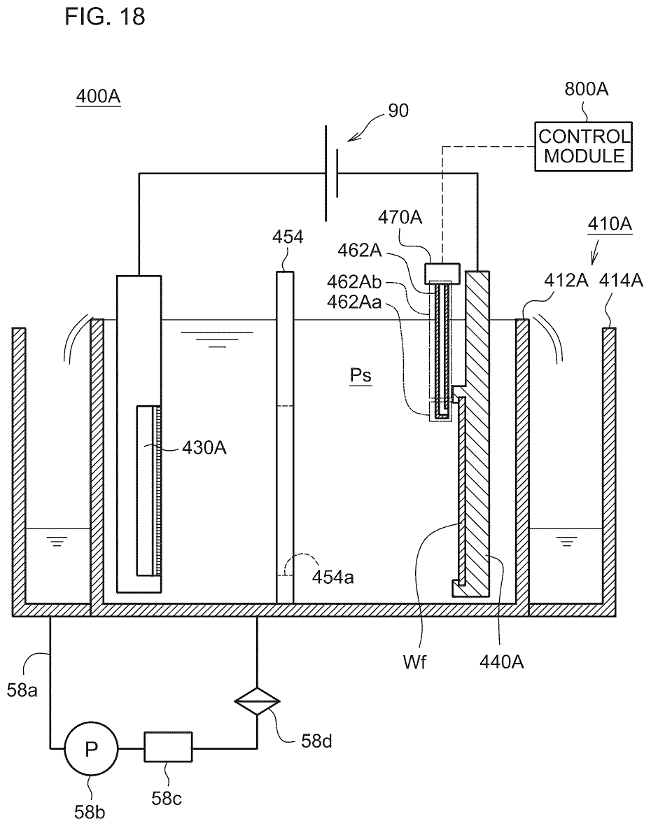

FIG. 18 is a cross-sectional view schematically showing a configuration of a plating module 400A in a third embodiment of the present invention. In the third embodiment, the substrate Wf is held so as to extend in the vertical direction, that is, the normal direction of the substrate Wf faces the horizontal direction. As shown in FIG. 18, the plating module 400A includes a plating bath 410A that holds the plating solution Ps therein, an anode 430A disposed in the plating bath 410A, and a substrate holder 440A. In the third embodiment, a rectangular substrate is used as an example of the substrate Wf, but similar to the first embodiment, the substrate Wf is not limited to a rectangular substrate and may be a circular substrate.

The anode 430A is disposed in an inner tank so as to be opposite the plating-target surface of the substrate Wf. The anode 430A is connected to a positive electrode of a power supply 90, and the substrate Wf is connected to a negative electrode of the power supply 90 via the substrate holder 440A. Upon applying a voltage between the anode 430A and the substrate Wf, a current flows through the substrate Wf, and a plating film (metal film) is formed on the surface of the substrate Wf in the presence of the plating solution Ps.

The plating bath 410A includes an inner tank 412A in which the substrate Wf and the anode 430A are disposed, and an overflow tank (outer tank) 414A adjacent to the inner tank 412A. The plating solution Ps in the inner tank 412A flows over a side wall of the inner tank 412A and into the overflow tank 414A.

One end of a plating liquid circulation line 58a is connected to a bottom portion of the overflow tank 414A, and the other end of the plating liquid circulation line 58a is connected to a bottom portion of the inner tank 412A. A circulation pump 58b, a thermostatic unit 58c, and a filter 58d are attached to the plating liquid circulation line 58a. When the plating solution Ps flows over the side wall of the inner tank 412A and flows into the overflow tank 414A, the inflowing plating solution is further returned to the inner tank 412A through the plating liquid circulation line 58a from the overflow tank 414A. Accordingly, the plating solution circulates between the inner tank 412A and the overflow tank 414A through the plating liquid circulation line 58a.

The plating module 400A further includes a regulation plate 454 that adjusts a potential distribution on the substrate Wf. The regulation plate 454 is disposed between the substrate Wf and the anode 430A, and has an opening 454a for limiting an electric field in the plating solution.

The plating module 400A is provided with a conduit 462A in the plating bath 410A. As an example, the conduit 462A can be formed from a resin such as polypropylene (PP) or polyvinyl chloride (PVC). Similar to the conduit 462 of the first embodiment or the second embodiment described above, the conduit 462A includes a first portion 462Aa including an opening end disposed in a region between the substrate Wf and the anode 430A, and a second portion 462Ab disposed in a region away from the region between the substrate Wf and the anode 430A. A potential sensor 470A is provided at the second portion 462Ab of the conduit 462A. A detection signal from the potential sensor 470A is input to the control module 800A.

In such a plating module 400A in the third embodiment, the control module 800A has the same functions as the control module 800 of the first embodiment or the control module 800T of the second embodiment. Therefore, the control module 800A can estimate the film thickness distribution of the plating film, based on a detection value from the potential sensor 470A. This allows the film thickness distribution of the plating film formed on the plating-target surface of the substrate Wf during the plating processing to be measured in real-time. The control module 800A can also adjust the plating conditions based on the film thickness of the plating film, similar to what is described in the first embodiment and the second embodiment.

It should be understood that modifications, additions, and improvements to the above embodiments can be made as appropriate without departing from the spirit and scope of the present invention. The scope of the present invention should be interpreted based on the description of the claims, and should be understood to include equivalents thereof.

1. A plating apparatus comprising:
a plating bath for accommodating a plating solution;
a substrate holder for holding a substrate;
an anode disposed in the plating bath so as to be opposite the substrate held by the substrate holder;
a sensor configured to measure a potential of the plating solution in a vicinity of a peripheral portion of the substrate held by the substrate holder; and
a state estimator configured to calculate, using the measured potential as input, a state estimate representing a current density at the peripheral portion of the substrate, based on an observation model and a state transition model, wherein
the state estimator is configured to execute computation based on the observation model by using a first trained neural network model or a first lookup table, and
when a state variable representing the current density at the peripheral portion is input, the first trained neural network model or the first lookup table is configured to output an estimate representing the potential of the plating solution in the vicinity of the peripheral portion.

2. The plating apparatus according to claim 1, wherein
the state transition model is a model that describes a temporal transition of the state variable representing the current density at the peripheral portion,
the observation model is a model that describes a relationship between the potential measured by the sensor and a state variable representing the current density at the peripheral portion, and
the state estimator is configured to calculate the state estimate by executing processing using a Kalman filter based on the state transition model and the observation model.

3. The plating apparatus according to claim 1, further comprising a data storage in which parameters of plating processing are stored, the parameters relating to at least one constituent element of a plating module including at least the plating solution, the plating bath, the substrate holder, and the anode, wherein
the first trained neural network model includes:
an input layer configured to accept parameter data provided from the data storage, and the state variable representing the current density at the peripheral portion;
an output layer configured to output the estimate representing the potential in the vicinity of the peripheral portion; and
an intermediate layer that couples the input layer and the output layer.

4. The plating apparatus according to claim 1, further comprising a data storage in which parameters of plating processing are stored, the parameters relating to at least one constituent element of a plating module including at least the plating solution, the plating bath, the substrate holder, and the anode, wherein
the first lookup table has a data structure in which a correspondence relationship is stored in advance, the correspondence relationship being between:
parameter data provided from the data storage as first input to the first lookup table;
the state variable representing the current density at the peripheral portion as second input to the first lookup table; and
the estimate representing the potential in the vicinity of the peripheral portion as output of the first lookup table.

5. The plating apparatus according to claim 1, further comprising a current density calculator configured to calculate, from the state estimate calculated by the state estimator, a distribution of a current density in an inner region of the substrate located inward of the peripheral portion of the substrate.

6. The plating apparatus according to claim 5, wherein the current density calculator is configured to calculate, from the state estimate calculated by the state estimator, the distribution of the current density in the inner region, by using a second trained neural network model or a second lookup table.

7. A plating apparatus comprising:
a plating bath for accommodating a plating solution;
a substrate holder for holding a substrate;
an anode disposed in the plating bath so as to be opposite the substrate held by the substrate holder;
a sensor configured to measure a potential of the plating solution in a vicinity of a peripheral portion of the substrate held by the substrate holder; and
a state estimator configured to calculate, from the measured potential, a state estimate representing a current density at the peripheral portion of the substrate; and
a current density calculator configured to calculate, from the state estimate calculated by the state estimator, a distribution of a current density in an inner region of the substrate located inward of the peripheral portion of the substrate, by using a trained neural network model or a lookup table.

8. The plating apparatus according to claim 7, further comprising a data storage in which parameters of plating processing are stored, the parameters relating to at least one constituent element of a plating module including at least the plating solution, the plating bath, the substrate holder, and the anode, wherein
the trained neural network model includes:
an input layer configured to accept parameter data provided from the data storage, and the state estimate provided from the state estimator;
an output layer configured to output the distribution of the current density in the inner region; and
an intermediate layer that couples the input layer and the output layer.

9. The plating apparatus according to claim 7, further comprising a data storage in which parameters of plating processing are stored, the parameters relating to at least one constituent element of a plating module including at least the plating solution, the plating bath, the substrate holder, and the anode, wherein
the lookup table has a data structure in which a correspondence relationship is stored in advance, the correspondence relationship being between:
parameter data provided from the data storage as first input to the lookup table;
the state estimate provided from the state estimator as second input to the lookup table; and
the distribution of the current density in the inner region as output of the lookup table.

10. A method of estimating a state in a plating apparatus including a plating bath for accommodating a plating solution, a substrate holder for holding a substrate, an anode disposed in the plating bath so as to be opposite the substrate held by the substrate holder, and a sensor configured to measure a potential of the plating solution in a vicinity of a peripheral portion of the substrate held by the substrate holder, the method comprising:
receiving data of the measured potential from the potential sensor; and
calculating, from the measured potential, a state estimate representing a current density at the peripheral portion of the substrate, based on an observation model and a state transition model, wherein
the calculating of the state estimate includes executing computation based on the observation model by using a first trained neural network model or a first lookup table, and
when a state variable representing the current density at the peripheral portion is input, the first trained neural network model or the first lookup table is configured to output an estimate representing the potential of the plating solution in the vicinity of the peripheral portion.

11. The method according to claim 10, further comprising calculating, from the calculated state estimate, a distribution of a current density in an inner region of the substrate located inward of the peripheral portion of the substrate, by using a second trained neural network model or a second lookup table.

12. A method of estimating a state in a plating apparatus including a plating bath for accommodating a plating solution, a substrate holder for holding a substrate, an anode disposed in the plating bath so as to be opposite the substrate held by the substrate holder, and a sensor configured to measure a potential of the plating solution in a vicinity of a peripheral portion of the substrate held by the substrate holder, the method comprising:
receiving data of the measured potential from the potential sensor; and
calculating, from the measured potential, a state estimate representing a current density at the peripheral portion of the substrate; and
calculating, from the calculated state estimate, a distribution of a current density in an inner region of the substrate located inward of the peripheral portion of the substrate, by using a trained neural network model or a lookup table.

13. A computer-readable non-transitory storage medium comprising a computer program including a plurality of instructions that when executed by a processor, causing the processor to implement the method according to claim 10.
